(19) United States
(12) Patent Application Publication
(10) Pub. No.: US 2016/0081943 A1
(43) Pub. Date: Mar. 24, 2016

(54) Process For Making A Core With An Active Coating

(71) Applicant: The Procter & Gamble Company, Cincinnati, OH (US)

(72) Inventors: Daren K. Anness, Loveland, OH (US); Richard John Dansereau, Cincinnati, OH (US); Nancy Lee Redman-Furey, Salem Township, OH (US)

(21) Appl. No.: 14/857,849

(22) Filed: Sep. 18, 2015

Related U.S. Application Data
(60) Provisional application No. 62/052,598, filed on Sep. 19, 2014.

(51) Int. Cl. A61K 9/50; A61K 31/137; B01J 2/16

(52) U.S. Cl. CPC A61K 9/5089; B01J 2/16; A61K 31/137; A61K 9/5078

(57) ABSTRACT

Forming a coated core by using a fluidized bed processor that discharges a spray containing atomized air and a coating solution where the coating solution contains an active. Then, wetting the core with the coating solution and drying the wetted cores to form coated cores. These steps can be repeated until an appropriate amount of active has been applied. The coated cores are visually perceived as smooth under a microscope with a total magnification of 40×.

### FIELD OF THE INVENTION

The present invention relates to methods of a coated core for use in a delayed release particles that contain soluble actives, such as phenylephrine, and more particularly a method of making delayed release dosage forms containing particles where the active coating is substantially smooth.

### BACKGROUND OF THE INVENTION

Particles can be coated with an active coating and a pH sensitive coating to make delayed release dosage forms. It can be difficult to determine the correct composition, thickness, and method to apply the active and pH sensitive coatings, especially when applying a soluble active like phenylephrine (PE).

One challenge with applying coatings is that it can be difficult to create particles, where the coating is a consistent thickness around the particle. In some cases, the active coating can appear spiked under 40× total magnification and when the pH sensitive coating is applied it can be thin at the top of the spikes. If the coating is uneven, some areas will dissolve before the particle reaches the desired portion of the digestive tract, prematurely releasing the soluble active.

Another challenge is that when a wet coating is applied, it can incorporate the previous layers. For instance, when a pH sensitive coating is applied directly to a core that is coated with phenylephrine hydrochloride (PE), the PE can leach into the pH sensitive coating, ultimately causing the pH sensitive coating to have PE incorporated into it, which will dissolve faster than the pH sensitive coating and ultimately cause early release of the PE via openings in the coating.

As such, there remains a need for a process for making cores with an active coating and/or a pH sensitive coating, where the cores are evenly coated and where the soluble active does not leach into the pH sensitive coating.

### SUMMARY OF THE INVENTION

A process for forming a coated core comprising: (a) in a fluidized bed processor, discharging a spray comprising atomized air and a coating solution wherein the coating solution comprises an active; (b) wetting a core with the coating solution; (c) drying the wetted cores to form coated cores; (d) repeating steps a, b, and c until the % active on the coated core is from about 8% to about 30%, by weight of the coated core; wherein the coated cores are substantially smooth as visually perceived under a microscope with a total magnification of 40×.

A process for forming a coated core comprising: (a) in a fluidized bed processor, discharging a spray comprising atomized air and a coating solution wherein the coating solution comprises phenylephrine or a salt thereof; (b) wetting a core with the coating solution; (c) drying the wetted cores to form coated cores; (d) repeating steps a, b, and c until the core has a % weight increase from about 15% to about 25%; and wherein the fluidized bed processor comprises an absolute humidity and wherein the absolute humidity is less than about 20 g of water vapor/kg of dry air.

### BRIEF DESCRIPTION OF THE DRAWINGS

FIG. 1A is a schematic of an immediate release particle;

FIG. 1B is a schematic of a delayed release particle;

FIG. 2 is a schematic of the Wurster processor;

FIG. 3 is a digital photograph of uncoated microcrystalline cellulose (MCC) cores under a total magnification of 40×;

FIG. 4A is a digital photograph of particles with an MCC core and an active coating where the particles contain 5% PE;

FIG. 4B is a digital photograph of particles with an MCC core and an active coating where the particles contain 6% PE;

FIG. 4C is a digital photograph of particles with an MCC core and an active coating where the particles contain 7% PE;

FIG. 4D is a digital photograph of particles with an MCC core and an active coating where the particles contain 8% PE;

FIG. 4E is a digital photograph of particles with an MCC core and an active coating where the particles contain 9% PE;

FIG. 4F is a digital photograph of particles with an MCC core and an active coating where the particles contain 10% PE;

FIG. 4G is a digital photograph of particles with an MCC core and an active coating where the particles contain 11% PE;

FIG. 5A is a digital photograph of particles with an MCC core and an active coating where the particles contain 5% PE;

FIG. 5B is a digital photograph of particles with an MCC core and an active coating where the particles contain 6% PE;

FIG. 5C is a digital photograph of particles with an MCC core and an active coating where the particles contain 7% PE;

FIG. 5D is a digital photograph of particles with an MCC core and an active coating where the particles contain 8% PE;

FIG. 5E is a digital photograph of particles with an MCC core and an active coating where the particles contain 9% PE;

FIG. 5F is a digital photograph of particles with an MCC core and an active coating where the particles contain 10% PE;

FIG. 5G is a digital photograph of particles with an MCC core and an active coating where the particles contain 11% PE;

FIG. 5H is a digital photograph of particles with an MCC core and an active coating where the particles contain 12% PE;

FIG. 6A is a digital photograph of particles with an MCC core and an active coating where the particles contain 5% PE;

FIG. 6B is a digital photograph of particles with an MCC core and an active coating where the particles contain 6% PE;

FIG. 6C is a digital photograph of particles with an MCC core and an active coating where the particles contain 7% PE;

FIG. 6D is a digital photograph of particles with an MCC core and an active coating where the particles contain 8% PE;

FIG. 6E is a digital photograph of particles with an MCC core and an active coating where the particles contain 9% PE;

FIG. 6F is a digital photograph of particles with an MCC core and an active coating where the particles contain 10% PE;

FIG. 6G is a digital photograph of particles with an MCC core and an active coating where the particles contain 11% PE;

FIG. 6H is a digital photograph of particles with an MCC core and an active coating where the particles contain 12% PE;

FIG. 7A is a digital photograph of particles with an MCC core and an active coating where the particles contain 5% PE;

FIG. 7B is a digital photograph of particles with an MCC core and an active coating where the particles contain 6% PE;

FIG. 7C is a digital photograph of particles with an MCC core and an active coating where the particles contain 7% PE;

FIG. 7D is a digital photograph of particles with an MCC core and an active coating where the particles contain 8% PE;

FIG. 7E is a digital photograph of particles with an MCC core and an active coating where the particles contain 9% PE;

FIG. 7F is a digital photograph of particles with an MCC core and an active coating where the particles contain 10% PE;

FIG. 7G is a digital photograph of particles with an MCC core and an active coating where the particles contain 11% PE;

FIG. 7H is a digital photograph of particles with an MCC core and an active coating where the particles contain 12% PE;

FIG. 8A is a digital photograph of particles with an MCC core and an active coating where the particles contain 5% PE;

FIG. 8B is a digital photograph of particles with an MCC core and an active coating where the particles contain 6% PE;

FIG. 8C is a digital photograph of particles with an MCC core and an active coating where the particles contain 7% PE;

FIG. 8D is a digital photograph of particles with an MCC core and an active coating where the particles contain 8% PE;

FIG. 8E is a digital photograph of particles with an MCC core and an active coating where the particles contain 9% PE;

FIG. 8F is a digital photograph of particles with an MCC core and an active coating where the particles contain 10% PE;

FIG. 8G is a digital photograph of particles with an MCC core and an active coating where the particles contain 11% PE;

FIG. 8H is a digital photograph of particles with an MCC core and an active coating where the particles contain 12% PE;

FIG. 9A is a digital photograph of particles with an MCC core and an active coating where the particles contain 5% PE;

FIG. 9B is a digital photograph of particles with an MCC core and an active coating where the particles contain 6% PE;

FIG. 9C is a digital photograph of particles with an MCC core and an active coating where the particles contain 7% PE;

FIG. 10A is a digital photograph of particles with an MCC core and an active coating where the particles contain 1% PE;

FIG. 10B is a digital photograph of particles with an MCC core and an active coating where the particles contain 3% PE;

FIG. 10C is a digital photograph of particles with an MCC core and an active coating where the particles contain 5% PE;

FIG. 10D is a digital photograph of particles with an MCC core and an active coating where the particles contain 7% PE;

FIG. 11A is a digital photograph of particles with an MCC core and an active coating where the particles contain 1% PE;

FIG. 11B is a digital photograph of particles with an MCC core and an active coating where the particles contain 3% PE;

FIG. 11C is a digital photograph of particles with an MCC core and an active coating where the particles contain 5% PE;

FIG. 11D is a digital photograph of particles with an MCC core and an active coating where the particles contain 7% PE;

FIG. 12A is a digital photograph of particles with an MCC core and an active coating where the particles contain 1% PE;

FIG. 12B is a digital photograph of particles with an MCC core and an active coating where the particles contain 3% PE;

FIG. 12C is a digital photograph of particles with an MCC core and an active coating where the particles contain 5% PE;

FIG. 12D is a digital photograph of particles with an MCC core and an active coating where the particles contain 7% PE;

FIG. 13A is a digital photograph of particles with an MCC core and an active coating where the particles contain 1% PE;

FIG. 13B is a digital photograph of particles with an MCC core and an active coating where the particles contain 3% PE;

FIG. 13C is a digital photograph of particles with an MCC core and an active coating where the particles contain 5% PE;

FIG. 13D is a digital photograph of particles with an MCC core and an active coating where the particles contain 7% PE; and

FIGS. 14A and 14B show exemplary images of the field of view used in the Smoothness Test Method.

### DETAILED DESCRIPTION OF THE INVENTION

Applying coatings, especially active coatings containing soluble actives or pH sensitive coatings, can be challenging. One challenge with applying these coatings is that it can be difficult to create particles, where the coatings are a consistent thickness around the particle. For instance, the active coating can be spiked, which means the coating is uneven and under a microscope has a spiked texture. This causes the pH sensitive coating, which is subsequently applied, to be uneven. While not wishing to be bound by theory, it is believed that the spikes are formed after the active coating is applied, when the particles move up the fluid bed and collide and instead of bouncing off each other, like dry particles would, they stick together and as the particles separate, the active coating can from a bridge, which eventually breaks and can form a spike. This can happen repeatedly and can create spiked particles. The spiked particles can be seen under a microscope at 40× total magnification. Then, when the pH sensitive coating is applied, it cannot be applied evenly and it can be especially thin at the top of the spike. The thinner portions of the pH sensitive coating will dissolve first in the digestive track and can cause the active to be prematurely released. This is especially problematic when the active is soluble, for instance a freely soluble active like PE, because the entire active can quickly exit the particle through the opening in the coating and enter the blood stream.

Another problem with spikes is that they can be friable and break off into a powder or small particles. These pieces can get incorporated into subsequent coatings, such as the pH sensitive coating and/or make the process less efficient by increasing yield loss. A pH sensitive coating that contains PE can dissolve faster than desired and ultimately cause the premature release of the PE via openings in the coating.

One way to limit the number of spikes that form is to modify the coating procedure to get the active coating to crystallize faster on the surface of the core. One way to achieve this is to coat the cores with a spray that contains a more concentrated active solution. However, increasing the concentration of the active in the spray can also increase the viscosity of the spray and the more viscous the spray the larger the droplets. Larger droplets can take longer to dry than small uniform droplets and the large droplets may not disperse to coat the particle as well. Another way to decrease the drying time is to spray the coating slower. However, this can significantly increase the coating time. The third way is to increase the heat by increasing the air flow or the air temperature. However, if the air temperature is too hot, this can degrade the active and if the air flow is too high then the coated cores can end up in the filter of the fluid bed.

Another challenge is that when a wet coating is applied, it can incorporate components from previously applied coatings. For instance, when a pH sensitive coating is applied directly to a core that is coated with phenylephrine hydrochloride (PE), the PE can leach into the pH sensitive coating, ultimately causing the pH sensitive coating to have PE incorporated into it. A pH sensitive coating that contains PE can dissolve faster than desired and ultimately cause the premature release of the PE.

One way to help mitigate this is to add a separation coating after applying the active coating to the cores with an active coating. A separation coating can help reduce the friability of PE and prevent PE from being incorporated into the pH sensitive coating.

As used herein, “binder” represents binders, which hold the ingredients together, commonly used in the formulation of pharmaceuticals. Non-limiting examples of binders can include polyvinylpyrrolidone, copolyvidone (cross-linked polyvinylpyrrolidone), povidone, polyethylene glycol, sucrose, dextrose, corn syrup, polysaccharides (including acacia, tragacanth, guar, and alginates), gelatin, sugar alcohols (including xylitol, sorbitol, maltitol and mannitol), and cellulose derivatives (including hydroxypropyl methylcellulose, hydroxypropyl cellulose, and sodium carboxymethylcellulose), and combinations thereof.

As used herein “delayed release” refers to a particle, a plurality of particles, or a dosage form where the drug active (or actives) are released at a time other than immediately following oral administration. In one example, a delayed release particle, plurality of particles, or dosage form has been deliberately modified such that the majority of the drug active that is contained in or on the particle, plurality of particles, or dosage form is released or absorbed into the blood plasma some period of time after administration. One advantage of a delayed release dosage form is that it can be formulated to release an active after a specified time period or upon encountering the proper environment (for example, release based on pH, enzymatic activity, or solubility). In one example, the delayed release particles have an enteric coating, which means that the particle coatings are pH sensitive and the benefit is not experienced by the user until the particle(s) or dosage form reaches certain regions of the intestine, specifically, the distal small intestine. In one example, a delayed release particle, plurality of particles, or a dosage form can be taken in combination with an immediate release particle, plurality of particles or dosage form. In one example, the dosage form or particle(s) do not deliver an active slowly over an extended duration of time, instead the particles can rapidly or immediately deliver an active after a delay period.

As used herein, “dissolve” refers to disintegrating, dispersing and/or passing into solution.

As used herein, “dose” or “dosage unit” refers to a dosage form containing an amount of a drug active suitable for administration on a single occasion, according to sound medical practice. The dosage form may include a variety of orally administered dosage forms. Non-limiting examples of dosage forms can include particles suspended in a liquid formulation, a solid in a gelatin or foam, or a solid dose in the form of a tablet, powder, granules, pellets, microspheres, nanospheres, particles, or nonpareils, and combinations thereof. In one example, the dosage form is a tablet or a capsule. Dosage forms can be orally administered and are typically swallowed immediately, slowly dissolved in the mouth, or chewed.

As used herein, “extended release” refers to a particle, a plurality of particles, or a dosage unit that that allows a reduction in dosing frequency as compared to that presented by a conventional dosage form, e.g., a solution or an immediate release dosage form. In one example, an extended release dosage form can be deliberately modified wherein the particle, plurality of particles, or dosage form is formulated in such a manner as to make the drug active available over an extended period of time following administration. One example of an extended release particle, plurality of particles or dosage form is a delayed release dosage form. Another example of an extended release particle, plurality of particles or dosage form can be pulsatile release dosage forms or particle(s).

As used herein, “immediate release” refers to a particle, a plurality of particles, or a dosage unit wherein no deliberate effort has been made to modify the release rate and in the case of capsules, tablets, and particles the inclusion of a disintegrating agent is not interpreted as a modification.

As used herein, “pulsatile release” refers to the phenylephrine being released at two or more distinct time periods following ingestion. In one example, the dosage form has a plurality of immediate release particles and a plurality of delayed release particles which results in an immediate release of the first pulse of phenylephrine after administration of the dosage form to the user and a second pulse when the delayed release particles enter the higher pH environment of the small intestine.

As used herein, “substantially free” means less than about 10%, less than 5%, less than 3%, less than 1%, less than about 0.5%, less than about 0.1%, or less than about 0.005% based on weight.

Any suitable process for applying and drying an active coating and additional coatings can be used.

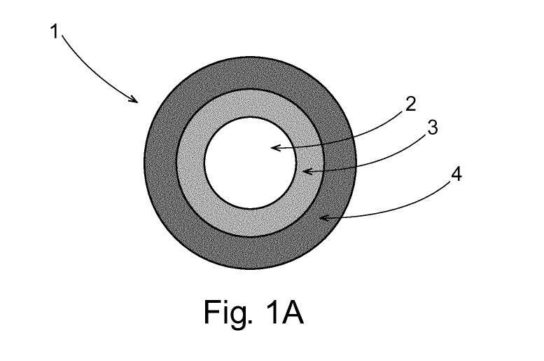

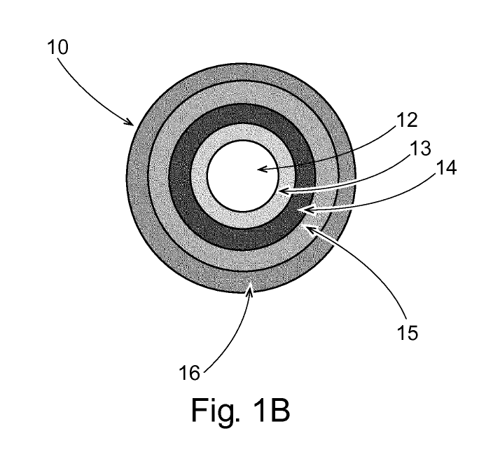

FIG. 1A shows a schematic of an immediate release particle 1. Immediate release particle 1 can comprise a core 2, an active coating 3, and optionally a separation coating 4. In one example, the immediate release particle can also contain an anti-caking coating. In one example, the active coating 3 can contain PE and can dissolve or start to dissolve after it reaches the stomach. The immediate release particles can be produced by the methods described herein or any suitable method. In one example, the immediate release dosage form is a powder, not a coated particle.

FIG. 1B shows a schematic of a delayed release particle 10 that can be produced by the methods described herein. Delayed release particle 10 comprises a core 12, active coating 13, optionally separation coating 14, pH sensitive coating 15, and optionally anti-caking coating 16. In one example, the active coating, the separation coating, the pH sensitive coating, and/or the anti-caking coating are substantially free of a binder.

The following examples were made using a Wurster type fluidized bed processor. Wurster processing can be utilized to provide uniform coatings to particles and cores. Wurster processing can be used to apply coatings including drug coatings and/or functional coatings.

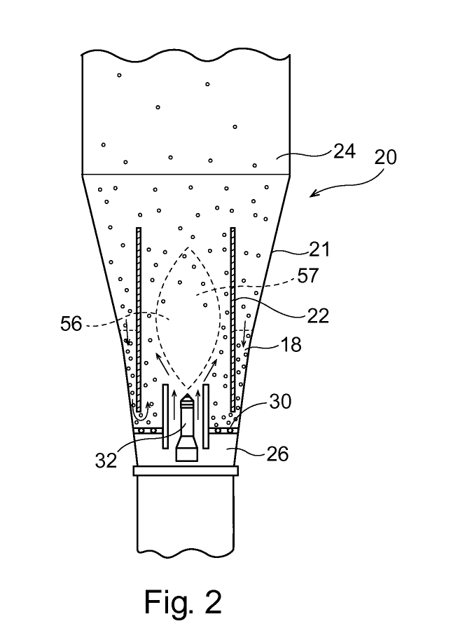

FIG. 2 shows a schematic of a representative Wurster type fluidized bed processor 20. The processor includes a product container section 21, an expansion chamber 24 into which the upper end of the product container section 21 opens, and a lower plenum 26 disposed beneath the product container. Product container section 21 and lower plenum 26 are separated by air distribution plate 18, which can have a plurality of openings 30 through which air or gas from the lower plenum 26 may pass into the product container section 21. The upper end of the expansion chamber 24 may open into a filter housing containing filters (not shown) disposed there above.

The spray nozzle assembly 32 discharges a spray of atomized air and coating solution, such as an active solution, which forms spray zone 56, into up-bed area 57. The coating solution can contain the components for the active coating and other functional coatings, including the pH sensitive coating. A pump can help control spray rate of the coating solution and the atomized air can help control the droplet size per vendor guidelines or process studies. The internal column 22 can direct the cores into spray zone 56 and internal column 22 can be raised or lowered to help control the flow rate of cores passing through spray zone 56. The internal column can generally be raised or lowered between about 25-50 mm.

Then, the wetted cores can be lifted out of spray zone 56, where the coated cores are dried. Drying conditions in the process are generally controlled by the inlet air temperature along with the inlet air dew point as the air flow rate is typically set to permit an acceptable “fountain” of cores above the internal column and below the filters. Dried cores can drift to expansion chamber 24, where they drop down to the down-bed where they can again pass up through spray zone 56 to receive additional coating. The process is repeated until the desired amount of coating has been applied. In one example, the openings 30 in air distribution plate 18 can be larger in the middle of the plate, for instance under internal column 22, as compared to the openings 30 in the exterior region of the plate. This design controls the flow of particles through the up-bed while permitting particles that have returned via the down-bed 18 enough movement to return to the up-bed for additional coating cycles.

One example of a fluidized bed can be found in U.S. Pat. No. 5,236,503.

Additional coatings, such as a pH sensitive coating, a separation coating, and an anti-caking coating can be applied using the fluid bed process, as described herein, or any other suitable process.

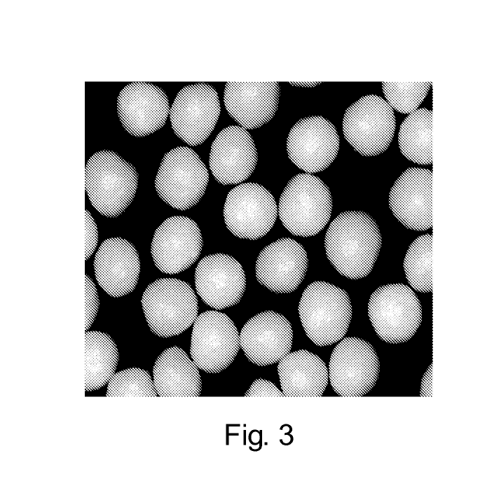

FIG. 3 is a digital photograph of uncoated MCC cores under a total magnification of 40×. The MCC cores are smooth and out-of-round.

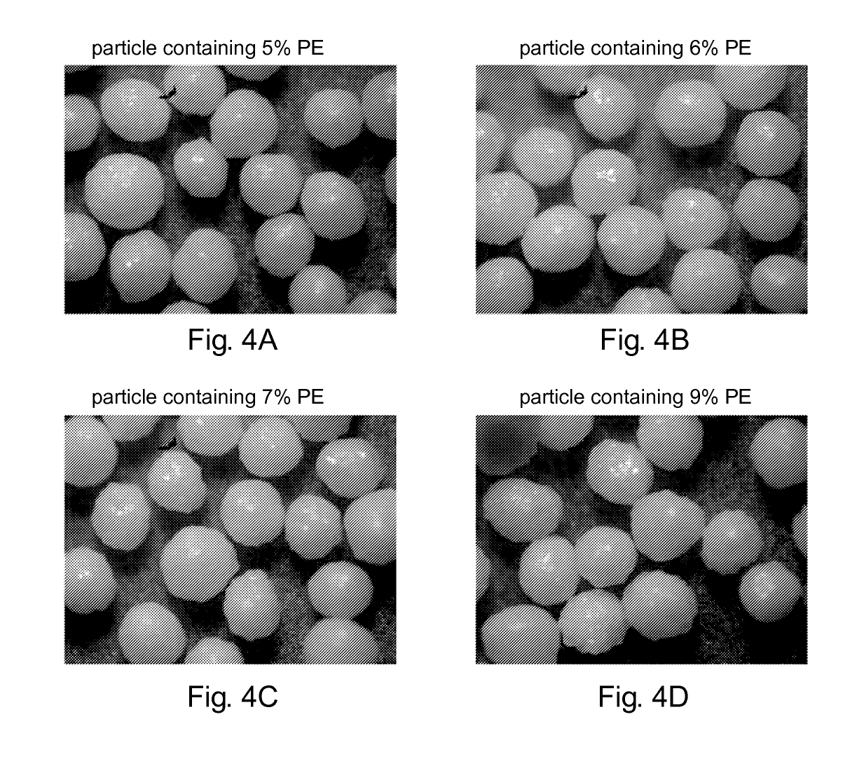

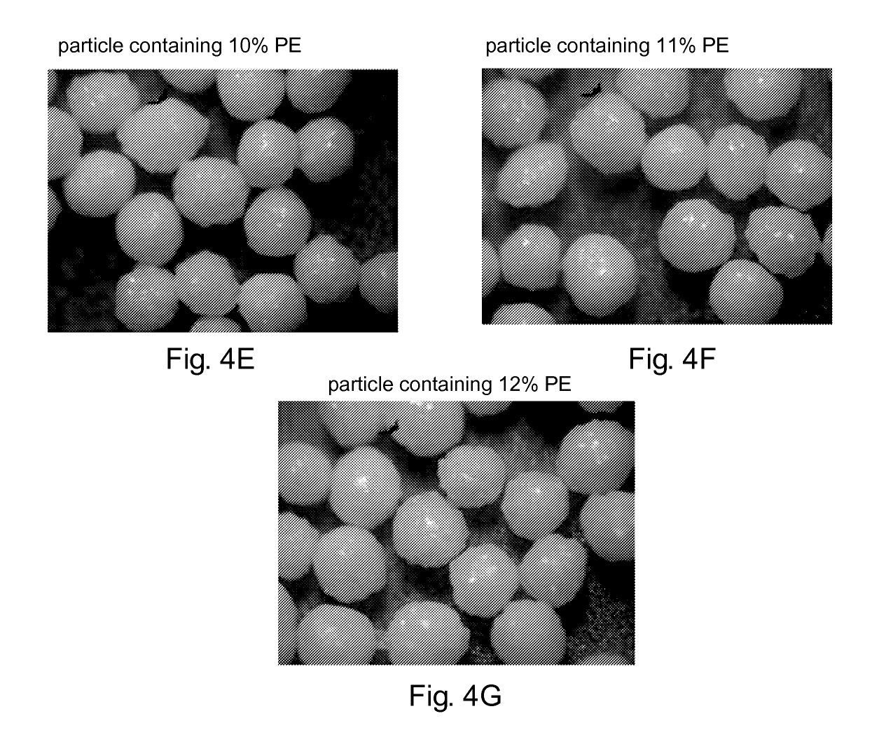

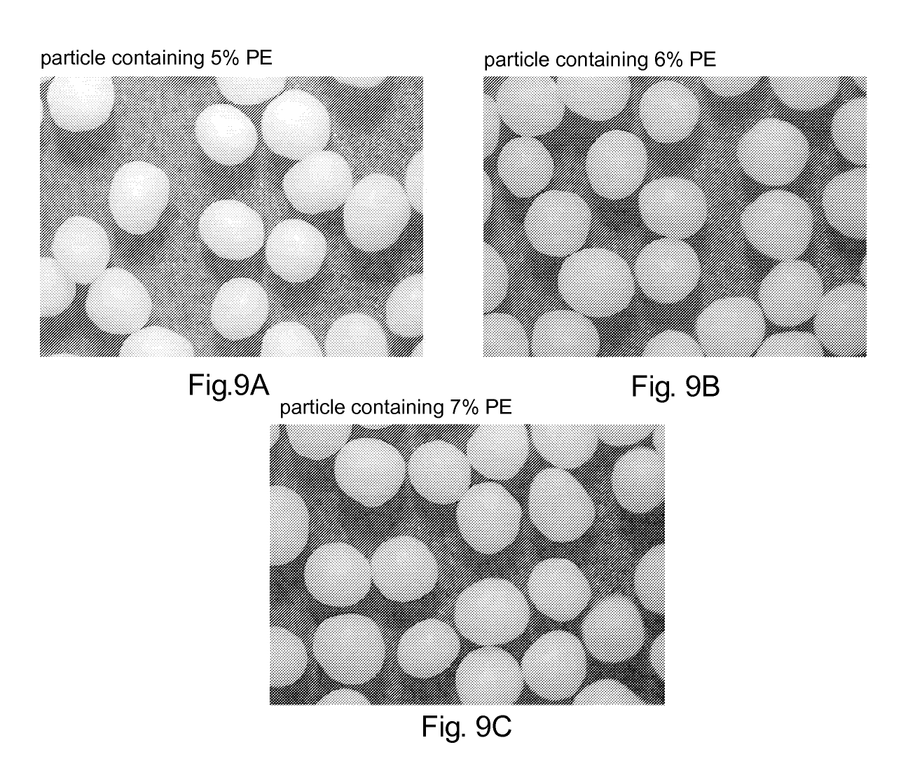

FIGS. 4 to 13 show digital photographs of MCC cores with an active coating under a total magnification of 40×. The amount of PE in the coating solution, the fluid bed product temperature, and the spray rate is varied for each set of FIGS. These variables can be seen in Table 1 below. All of the examples in FIGS. 4 to 8 were processed with a Wurster size of nine inches and a starting batch size of 7000 g. FIG. 9 is a larger batch and used a Wurster size of 18 inches and a 52.5 kg starting batch size. The spray rate can depend on the size of the fluid bed and the starting batch size and can be scaled according to a scaling factor of the equipment being used. The water rate is the amount of the spray rate that is water-based. It can be determined by calculating the rate that PE and ethanol (if present) are applied to the cores and then subtracting this value from the spray rate of the coating solution. The active coating solution can be made by dissolving the appropriate amount of PE in USP water and adding ethanol (if desired) at ambient temperature.

Absolute humidity reflects the “dryness” of the particles in the column by encompassing the optimization of water rate, dew point, air flow rate, and solution composition. Absolute humidity in Table 1 reflects the absolute humidity at the top of the Wurster column where it is assumed that the product temperature equals the outlet air temperature, that is the temperature of the product and air is at equilibrium. Absolute humidity is estimated as the sum of the inlet air absolute humidity plus the contribution of drying (from the water rate). In one example, the inlet air absolute humidity at 10° C. dew point is 7.72 g of water vapor/kg of dry air. “Section 12: Psychrometry” Perry's Chemical Engineers' Handbook, 8^{th} Edition, Mc Graw-Hill Education, pages 12-1 to 12-17 contains charts and equations that are generally accepted in the industry to allow for calculation of absolute humidity. The contribution from drying is simply the water rate dividing by the air flow rate (also converting the air flow to dry air flow and converting units including using density of air).

In one example, if the conditions in the Wurster column are too wet (i.e. the absolute humidity is too high) it can lead to spiked particles. However, if the conditions are too dry (i.e. the absolute humidity is too low) the spray will be slow. If the process is too slow, it is not only inefficient, but it could cause the particles to become too warm and possibly degrade the active and it could also cause the particles to become friable.

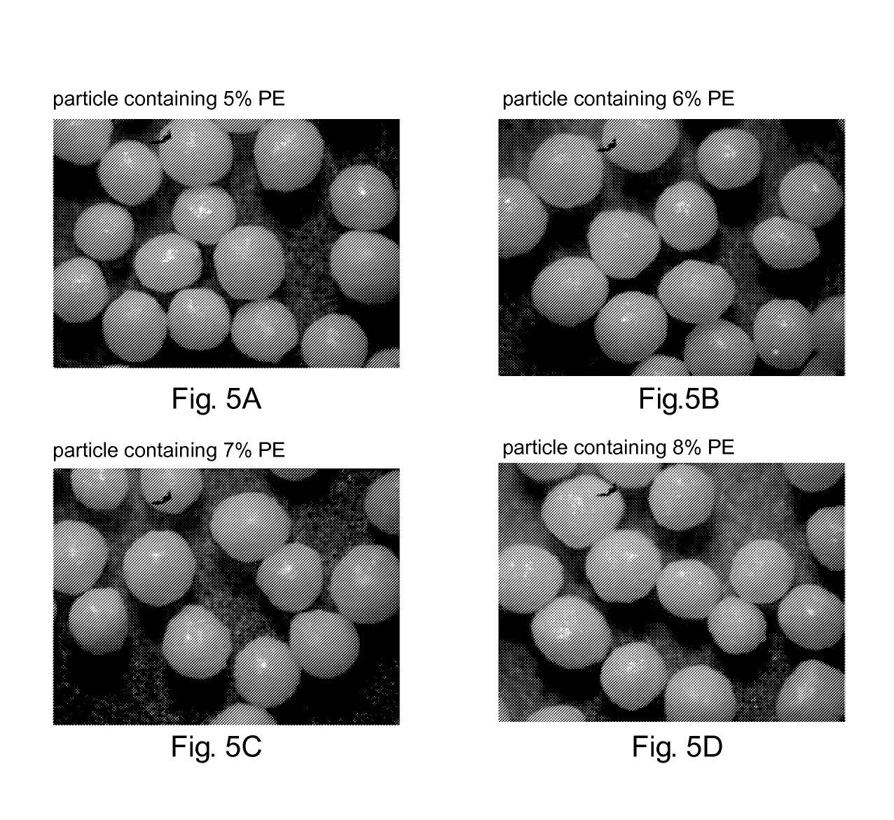

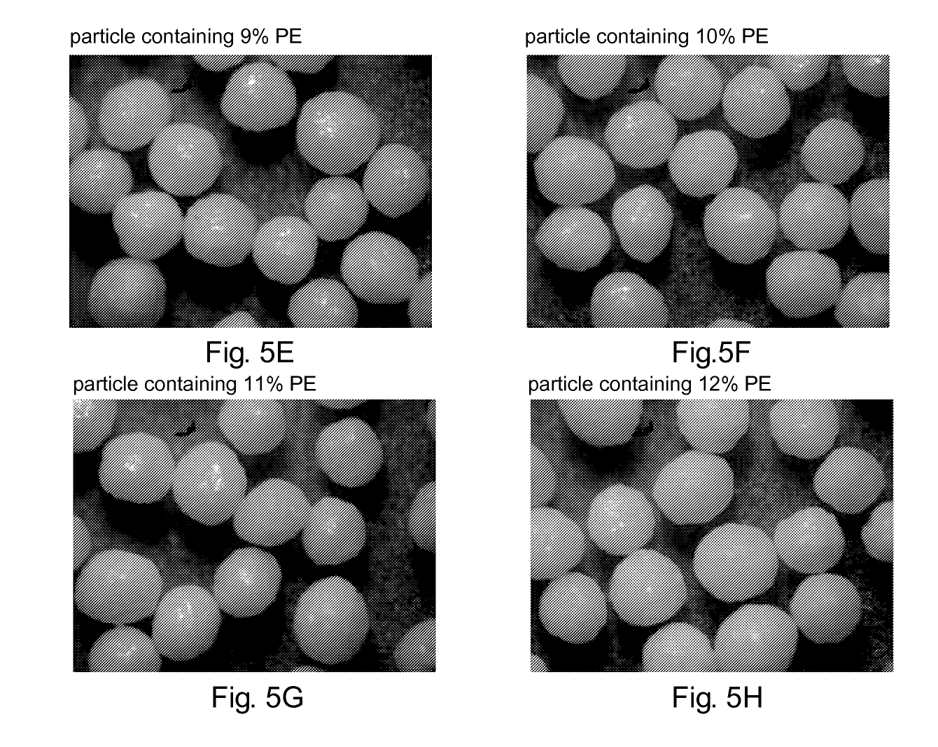

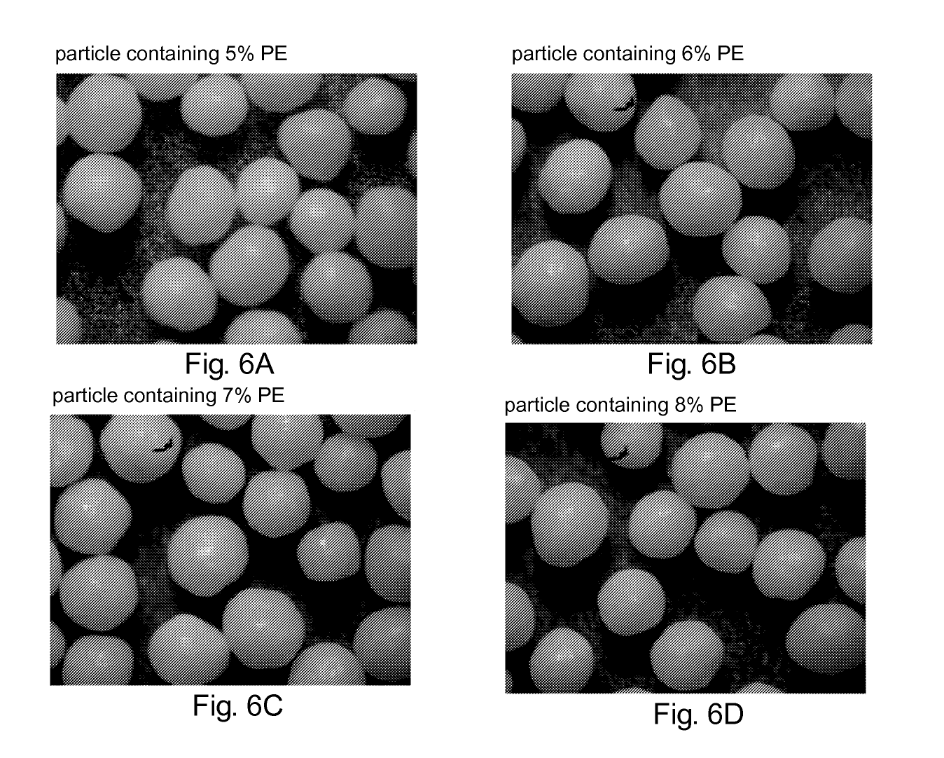

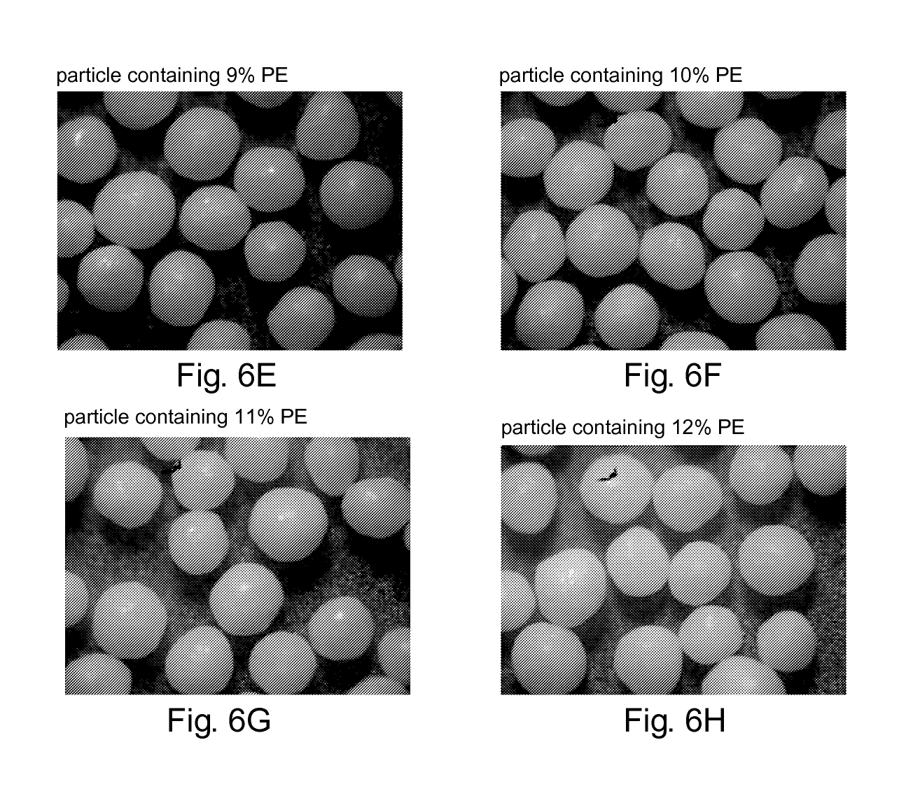

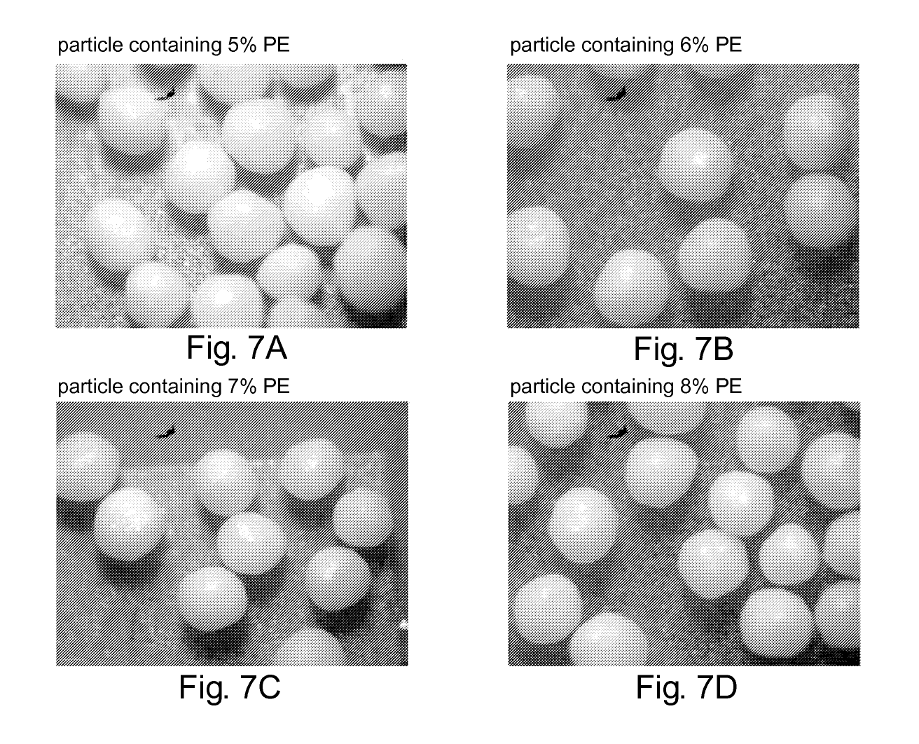

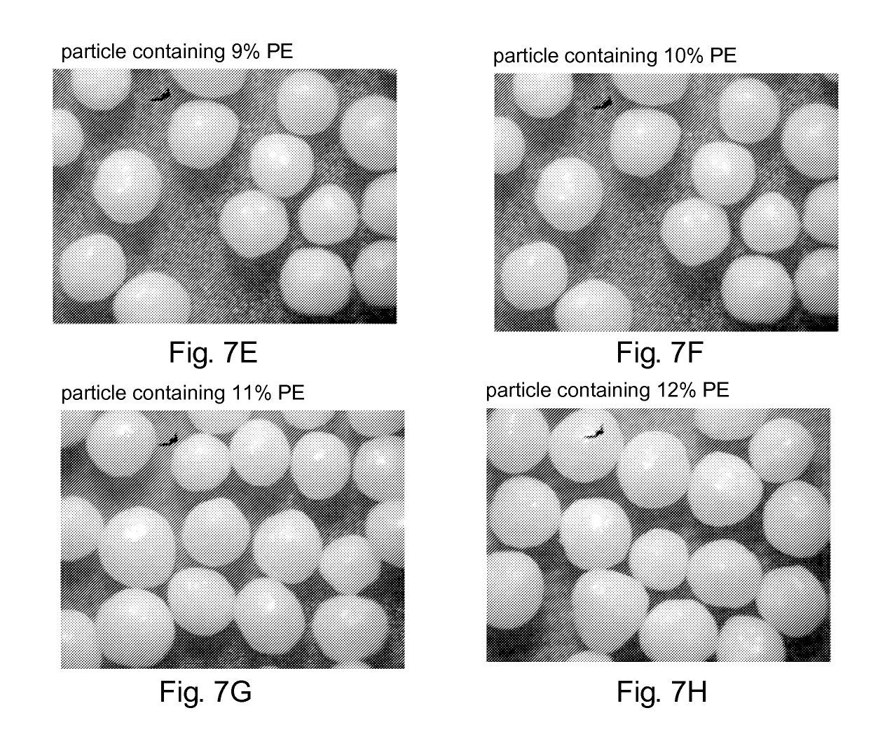

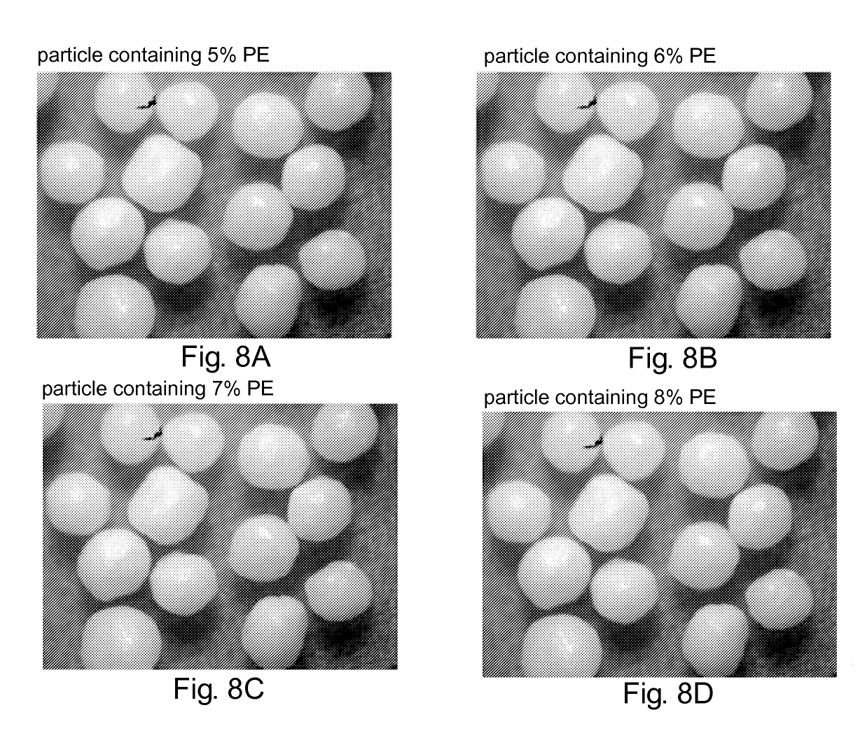

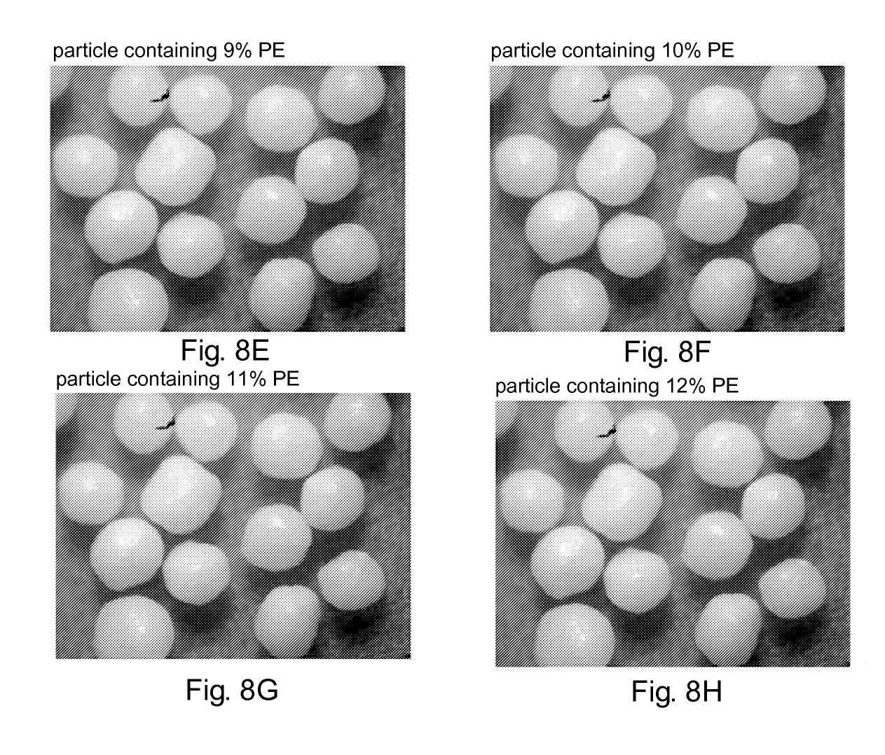

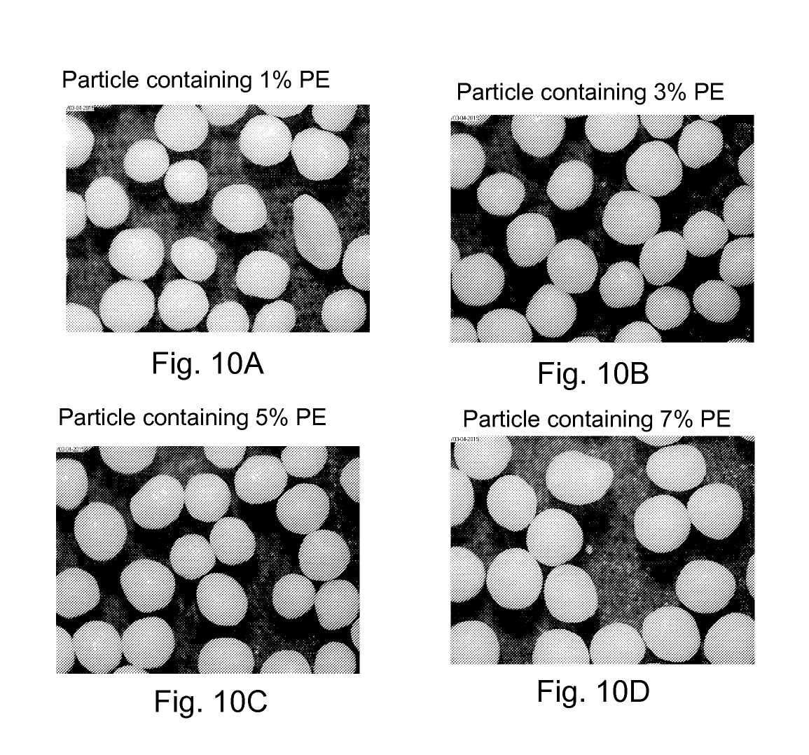

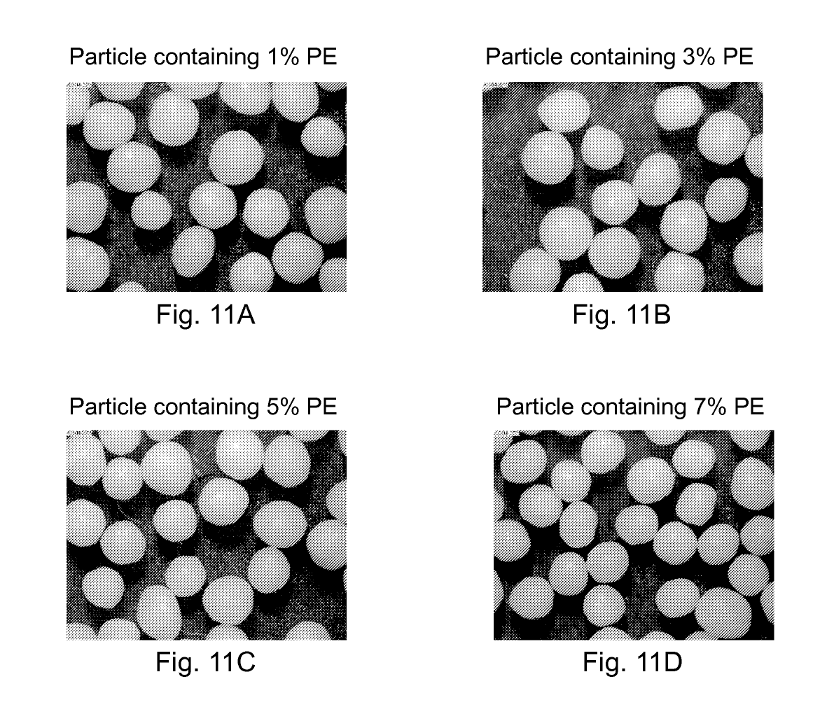

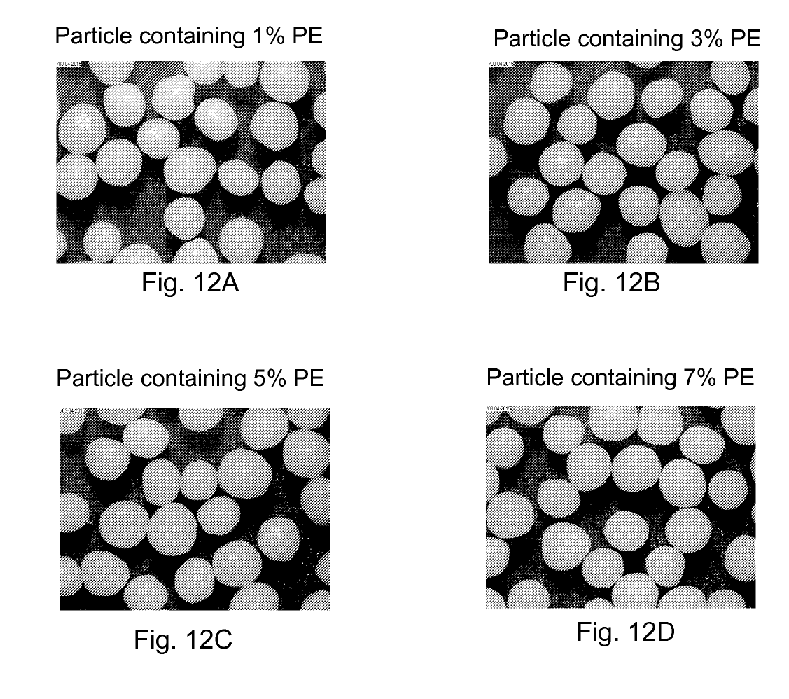

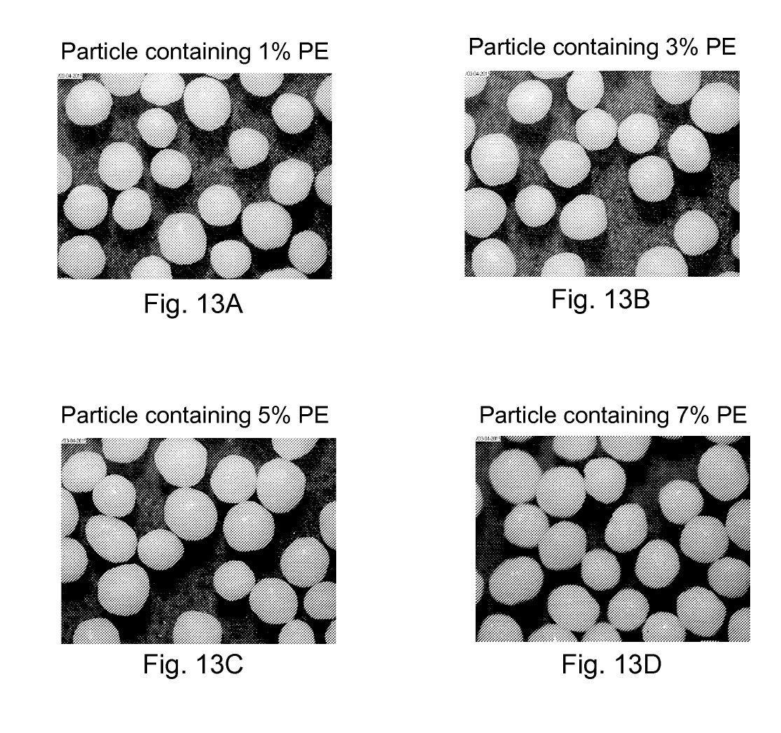

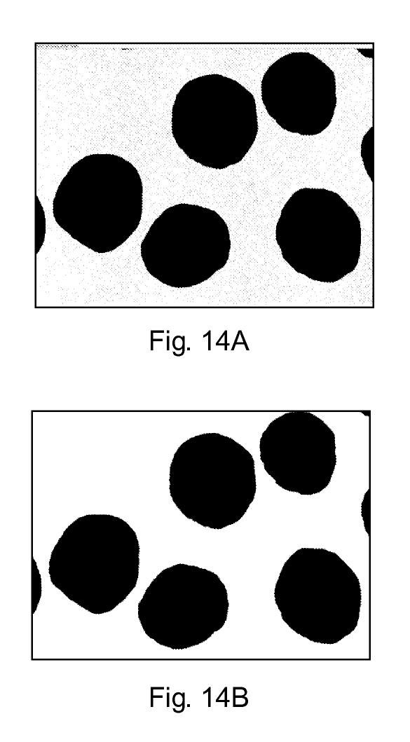

TABLE 1

| | Active | Fluid Bed | Spray Rate | | Air | Inlet | Inlet | |
|---|---|---|---|---|---|---|---|---|
| | Coating | Product | (g/min) of | Water | Flow | Air | Air Dew | Absolute |
| | Solution | Temp. | the coating | Rate | Rate | Temp. | Point | Humidity |
| | Composition | (° C.) | solution | (g/min) | (cfm) | (° C.) | (° C.) | (g H2O/kg air) |
| FIGS. 4A to | 10% PE and | 40 | 60 | 51.6 | 170 | 73 | 10 | 18.36 |
| 4G | 4% ethanol | | | | | | | |
| FIGS. 5A to | 15% PE | 40 | 40 | 34 | 170 | 63 | 10 | 14.63 |
| 5H | | | | | | | | |
| FIGS. 6A to | 30% PE | 60 | 20 | 14 | 170 | 75 | 11 | 11.04 |
| 6H | | | | | | | | |
| FIGS. 7A to | 30% PE | 40 | 20 | 14 | 170 | 51 | 10 | 10.51 |
| 7H | | | | | | | | |
| FIGS. 8A to | 22.5% PE | 40 | 26.67 | 20.67 | 170 | 54 | 10 | 11.89 |
| 8H | | | | | | | | |
| FIGS. 9A to 9C | 22.5% PE | 40 | 115 | 89.1 | 610 | 48 | 10 | 12.74 |
| FIGS. 10A to | 22.5% PE | 40 | 115 | 89.1 | 610 | 58 | 10 | 12.74 |
| 10D | | | | | | | | |
| FIGS. 11A to | 22.5% PE | 40 | 155 | 120.1 | 610 | 62 | 10 | 14.53 |
| 11D | | | | | | | | |
| FIGS. 12A to | 22.5% PE | 40 | 115 | 89.1 | 510 | 60.5 | 10 | 13.75 |
| 12D | | | | | | | | |
| FIGS. 13A to | 22.5% PE | 40 | 143 | 110.8 | 530 | 63 | 10 | 14.95 |
| 13D | | | | | | | | |

FIG. 3 is a digital photograph of uncoated microcrystalline cellulose (MCC) cores under a total magnification of 40×. The cores are smooth and round, however most of the cores are out-of-round.

FIGS. 4 to 13 are digital photographs of MCC cores with different thickness of active coatings. As shown in Table 1 above, each set of FIGS. had slightly different processing conditions. In some examples, as the amount of active coating increased, the coated cores can become more uneven and eventually can become spiky. At a certain point, the coated cores become too spiky and it can become difficult to evenly apply a pH sensitive coating.

FIGS. 4 to 13 can be compared to the cores in FIG. 3 to determine if the coated cores are substantially smooth, as visually perceived under a microscope with a total magnification of 40×. As used herein, “visually perceived under a microscope” means that a human viewer can visually discern that the coated core is smooth and the surface has an appearance that is substantially similar to the cores, as shown in FIG. 3, under a properly focused microscope with a total magnification of 40×.

In another example, smoothness can be determined by the Smoothness Test Method, as described hereafter. In one example, the particles can have a mean circularity from about 0.70 to about 1 as determined by the Smoothness Test Method, in another example from about 0.75 to about 1, in another example from about 0.8 to about 1, in another example from about 0.85 to about 1, in another example from about 0.90 to about 1, and in another example from about 0.95 to about 1. In another example particles can have a mean circularity from about 0.72 to about 0.95 as determined by the Smoothness Test Method, to about 0.78 to about 0.93, and from about 0.82 to about 0.89.

The examples in FIGS. 4A to 4G are digital photographs of cores that were coated with an active coating solution containing 10% PE and 4% ethanol. The spray rate, for this batch and Wurster size was also faster than the examples in FIGS. 5 to 8. FIG. 4A could be acceptable for smoothness, although they are clearly not ideal. However, FIGS. 4B to 4G are all too spiked and are not substantially smooth and are not recommended for use as delayed release particles. Under these processing conditions it is not recommended to add ethanol to the active coating solution in order to help the coating dry faster. However, in another example, probably under different processing conditions, it may be advantageous to add ethanol. While not wishing to be bound by theory, the coated cores in this example may be too spiky because the water content is too high.

The examples in FIGS. 5A to 5H are digital photographs of cores that were coated with an active coating solution containing 15% PE and a spray rate of 40 g/min Although these FIGS. are smoother than the corresponding examples in FIGS. 4A to 4G, the coated cores are only marginally better and may not be ideal for use in delayed release particles. While not wishing to be bound by theory, the coated cores in this example may be too spiky because the water rate is still too high.

The examples in FIGS. 6A to 6H appear ideal, in terms of smoothness, as the coated cores are substantially smooth, as visually perceived under a microscope with a total magnification of 40×. In these examples, the active coating solution composition had 30% PE and was sprayed at a rate of 20 g/min.

The examples in FIGS. 7A to 7H also appear ideal, in terms of smoothness, as the coated cores are substantially smooth, as visually perceived under a microscope with a total magnification of 40×. These examples, use the same active coating solution with 30% PE and spray rate of 20 g/min as the examples in FIGS. 6A to 6H. However, the fluid bed product temperature is 20° C. lower than the examples in FIGS. 6A and 6H. Lower temperature can be advantageous as it can be less expensive to use a lower temperature and it can also better preserve actives, especially if the actives can be sensitive to heat. It is surprising that similar results can be achieved at a lower temperature.

The examples in FIGS. 8A to 8H also appear ideal, in terms of smoothness, as the coated cores are substantially smooth, as visually perceived under a microscope with a total magnification of 40×.

The examples in FIGS. 9A to 9C also appear ideal, in terms of smoothness, as the coated cores are substantially smooth, as visually perceived under a microscope with a total magnification of 40×. These examples are run with a larger batch size and a larger Wurster.

The examples in FIGS. 10A to 10D, 11A to 11D, 12A to 12D, and 13A to 13D also appear ideal, in terms of smoothness, as the coated cores are substantially smooth, as visually perceived under a microscope with a total magnification of 40×.

In one example, the active coating solution contains from about 5% to about 50% PE, in another example from about 10% to about 40% PE, in another example from about 12% to about 35% PE, in another example from about 15% to about 30% PE, and in another example from about 20% to about 25% PE. In one example, the coating solution does not contain ethanol.

In one example, the spray rate of the active coating solution is from about 10 g/min to about 70 g/min, in another example from about 15 g/min to about 60 g/min, in another example from about 18 g/min to about 45 g/min, in another example from about 20 g/min to about 40 g/min, and in another example from about 22 g/min to about 30 g/min. In another example, the spray rate of the active coating solution is from about 40 g/min to about 200 g/min, in another example from about 50 g/min to about 150 g/min, and in another example from about 80 g/min to about 120 g/min. In another example, the spray rate can be from about 180 g/min to about 650 g/min, in another example from about 250 g/min to about 550 g/min, in another example from about 300 g/min to about 500 g/min, and in another example from about 325 g/min to about 450 g/min. In another example, the spray rate can be from about 390 to about 1350 g/min, in another and in another example from about 500 g/min to about 1100 g/min, in another example from about 600 g/min to about 900 g/min, and in another example from about 700 g/min to about 1000 g/min. In one example the spray rate may not be greater than about 650 g/min and in another example the spray rate may not be greater than about 1350 g/min.

In one example, the water rate is from about 5 g/min to about 55 g/min, in another example from about 10 g/min to about 40 g/min, in another example from about 12 g/min to about 35 g/min, and in another example from about 14 g/min to about 25 g/min.

In one example, the dew point can be between about 3° C. to about 25° C., in another example from about 5° C. to about 20° C., and in another example from about 7° C. to about 15° C., in another example from about 9° C. to about 13° C., in another example from 8° C. to 12° C., in another example from 9° C. to 11° C., and in another example about 10° C.

In one example the inlet air temperature can be from 35° C. to about 90° C., in another example from about 40° C. to about 80° C., in another example from about 45° C. to about 80° C., in another example from about 48° C. to about 75° C., in another example from about 50° C. to about 70° C., and in another example from about 52° C. to about 65° C. In another example the inlet air temperature can be from about 45° C. to about 55° C.

In one example, the fluid bed product temperature can be from about 25° C. to about 80° C., in another example from about 30° C. to about 70° C., in another example from about 35° C. to about 65° C., and in another example from about 40° C. to about 60° C. In another example, the fluid bed temperature is less than about 60° C., in another example less than about 50° C., and in another example less than about 45° C.

In one example, the absolute humidity is from about 8 g of water vapor/kg of dry air to about 30 g of water vapor/kg of dry air, in another example from about 12 g of water vapor/kg of dry air to about 28 g of water vapor/kg of dry air, in another example from about 14 g of water vapor/kg of dry air to about 25 g of water vapor/kg of dry air, in another example from about 16 g of water vapor/kg of dry air to about 22 g of water vapor/kg of dry air, in another example from about 15 g of water vapor/kg of dry air to about 20 g of water vapor/kg of dry air, and in another example from about 17 g of water vapor/kg of dry air to about 19 g of water vapor/kg of dry air. In another example, the absolute humidity is greater than about 10 g of water vapor/kg of dry air, in another example greater than about 13 g of water vapor/kg of dry air, in another example greater than about 14 g of water vapor/kg of dry air, in another example greater than about 15 g of water vapor/kg of dry air, in another example greater than about 16 g of water vapor/kg of dry air, in another example greater than about 17 g of water vapor/kg of dry air, and in another example greater than about 18 g of water vapor/kg of dry air. In another example the absolute humidity is less than about 30 g of water vapor/kg of dry air, in another example less than about 27 g of water vapor/kg of dry air, in another example less than about 24 g of water vapor/kg of dry air, in another example less than about 21 g of water vapor/kg of dry air, in another example less than about 20 g of water vapor/kg of dry air, in another example less than about 19 g of water vapor/kg of dry air, in another example less than about 18 g of water vapor/kg of dry air, and in another example less than about 17 g of water vapor/kg of dry air.

In another example, the % of active in the coated core after the active coating is applied can be from 2% to about 20%, in another example from about 5% to about 15%, in another example from about 7% to about 12%, in another example from about 8% to about 10%, and in another example from about 7% to about 9%. In another example, the % of active in the coated core after the active coating is applied can be greater than about 5%, in another example greater than about 6%, in another example greater than about 7%, in another example greater than about 8%, in another example greater than about 9%, in another example greater than about 10%, in another example greater than about 11%, and in another example greater than about 12%. In yet another example, the % of active in the coated core after the active coating is applied can be less than about 25%, in another example less than about 20%, in another example less than about 15%, in another example less than about 12%, and in another example less than about 10%. In another example the % of active in the coated core after the active coating is applied can be from about 8% to about 30%, in another example from about 10% to about 25%, in another example from about 12% to about 20%, and in another example from about 13% to about 18%.

In one example, the ratio of water rate to spray rate of the coating solution is less than about 0.85, in another example less than about 0.8, and in another example less than about 0.88. In another example, the ratio of water rate to spray rate of the coating solution is from about 0.5 to about 0.9, in another example from about 0.6 to about 0.86, in another example from about 0.7 to about 0.8, and in another example from about 0.75 to about 0.78.

The process described herein can be used with any soluble active. In one example, the active can be at least soluble, where the part of the solvent required per part of solute is from about 10 to about 30. In another example, the active can be at least freely soluble, where the part of solvent required per part of solute is from about 1 to about 10. In another example, the active can be very soluble, where the part of solvent required per part of solute is less than about 1. In another example, the part of solvent required per part of solute can be less than about 30, in another example less than about 20, in another example less than about 15, in another example less than about 10, in another example less than about 8, in another example less than about 6, and in another example less than about 5. In another example, the part of solvent required per part of solute is from about 0.1 to about 20, in another example from about 0.5 to about 15, in another example from about 1 to about 10, and in another example from about 2 to about 8. The solubility can be determined by the method described in Etzweiler, Franz., Erwin. Senn, and Harald W. H. Schmidt “Method for Measuring Aqueous Solubilities of Organic Compounds.” Analytical Chemistry (1995): 655-58. 1 Feb. 1995. In one example, the active can be selected from the group consisting of phenylephrine hydrochloride, pseudophedrine hydrochloride, phenylpropanolamine, ibuprofen sodium, and combinations thereof.

In one example, the active coating, the separation coating, the pH sensitive coating, and/or the anti-caking coating are substantially free of a binder. While not wishing to be bound by theory, it is believed that the binder inhibits the rate of crystallization, which can increase the unevenness and spikiness of the cores. In one example, the active coating is substantially free of a binder. In one example the active coating is substantially free of polyvinyl alcohol. In another example the active coating can contain polyvinyl alcohol.

In another example, the active coating, the separation coating, the pH sensitive coating, and/or the anti-caking coating can include a binder.

The method of the present invention can be used to create immediate release particles and/or delayed release particles that can be incorporated into a dosage form. In one example, a multi-particle, oral dose form designed for an immediate release of PE followed by one or more delayed pulses. The dosage form can be a tablet, a sachet, or a capsule, containing PE which can be administered every 6, 8, or 12 hours to provide extended congestion relief to a patient.

In one example, the immediate release particle can have a core, a PE coating, and optionally a separation and/or an anti-caking coating and the delayed release particle can comprise a core, a PE coating, optionally a separation coating, a pH sensitive coating, and optionally an anti-caking coating.

The core can contain any pharmaceutically suitable material. Non-limiting examples of core materials can consist of microcrystalline cellulose, sugars, starches, polymers, and combinations thereof. In one example, the core can be microcrystalline cellulose spheres marketed under the tradename “Cellets®” available from Glatt® Air Techniques Inc., Ramsey, N.J.. In one example, the microcrystalline cellulose spheres can have a diameter of about 500 μm to about 710 μm and a bulk density of about 0.7 g/cc to about 0.9 g/cc.

In one example, the immediate release particles and/or the delayed release particles can have a separation coating. Non-limiting examples of separation coatings can include talc, polyvinyl alcohol-polyethylene glycol graft co-polymer (commercially available as Kollicoat® IR, from BASF, Tarrytown, N.J.), hydroxypropyly methylcellulose, hydroxypropyl cellulose, polyvinylpyrrolidine, and combinations thereof. In another example, the separation coating can be a pH independent polymer. In one example, the separation coating can contain polyvinyl alcohol. In one example, the separation coating can be added as a solution that is from about 5% to about 25% solids, in another example from about 7.5% to about 15% solids. In one example, the anti-caking coating can be sprayed or added as dry powder onto the delayed release particles to prevent the particles from sticking together during storage. In another example, the immediate release particles can have an anti-caking coating. If the particles stick together, this can cause uneven dissolution, which alters the carefully timed release of the phenylephrine. The anti-caking coating can be any material that prevents the particles from sticking together. In one example, the anti-caking coating can be clear and in another example the anti-caking coating can be translucent. In another example, the anti-caking coating can be opaque. In another example, the anti-caking coating can be a white powder. In another example, the anti-caking coating can contain a color. In one example, the anti-caking coating can contain a fine particulate that has a high relatively high surface area and is insoluble in water. In one example the surfaces area is greater than about 100 m²/g, in another example greater than about 150 m²/g, in another example greater than about 175 m²/g, and in another example greater than about 200 m²/g. In one example, the weight percent (wt. %) increase of the particle after the anti-caking coating is added can be from about 0.1% to about 5%, in another example from about 0.15% to about 3%, and in another example from about 0.2% to about 2%.

Non-limiting examples of anti-caking coatings can include talc, sodium ferrocyanide, potassium ferrocyanide, calcium carbonate, magnesium carbonate, silicon dioxide, hydrophilic fumed silica (commercially available as Aerosil® 200, Evonik Industries, Parsippany, N.J., precipitated silica, sodium aluminosilicate, and combinations thereof. In one example, the anti-caking coating contains hydrophilic fumed silica. In another example, the anti-caking coating can contain a thin aqueous coating based on glycerol monostearate and/or hydroxypropyl methylcellulose. In another example, the anti-caking coating can contain polyvinyl alcohol, and/or polyvinyl alcohol-polyethylene glycol graft copolymer (commercially available as Kollicoat® IR, BASF, Tarrytown, N.J.).

In one example, the delayed release particles can contain a pH sensitive coating which means that the coating dissolves when it is immersed in a particular pH, which can be basic or acidic. In one example the pH sensitive coating is an enteric coating. It can be important for the coating to be the appropriate thickness or appropriate weight percentage. If the coating is too thin or the weight percentage is too low, then the phenylephrine can be released prematurely and the lag time will be shorter than required. One problem with releasing the phenylephrine prematurely is that the doses can be too close together and the user will not have a sustained level of uncongugated phenylephrine for the intended duration.

If the coating is too thick or if the weight percentage is too high, then the phenylephrine can be released in the proximal large intestine and/or the distal large intestine, which can mean that the phenylephrine is released suboptimally with respect to achieving the intended 6-12 hour duration of dosing. If the phenylephrine is released too distally in the small intestine then there may not be enough time for the phenylephrine to enter the blood stream before entering the colon and/or the phenylephrine may not be completely dissolved. Furthermore, if the phenylephrine is released in the large intestine, there can be minimal absorption due to the reduced surface area of the large intestine as compared to the small intestine. While not wishing to be bound by theory, the colon may not have enough liquid to allow the dissolution of phenylephrine and thus systemic absorption. Therefore significant dissolution of the dose form and active can occur prior to migration into the colon.

The weight percent (wt. %) increase of the particle after the pH sensitive coating is added can be from about 15 wt. % to about a 65 wt. % increase, in another example from about a 25 wt. % to about a 55 wt. %, and in another example from about a 35 wt. % to about a 45 wt. %.

In another example, the wt. % increase after the pH sensitive coating is added can be from about 25 wt. % to about a 75 wt. % increase, in another example from about a 35 wt. % to about a 45 wt. %, and in another example from about a 45 wt. % to about a 55 wt. %.

In another example, the wt. % increase after the pH sensitive coating is added can be from about 40 wt. % to about a 80 wt. % increase, in another example from about a 50 wt. % to about a 75 wt. %, and in another example from about a 55 wt. % to about a 65 wt. %.

In another example, the wt. % increase after the pH sensitive coating is added is from 20 wt. % to about 60 wt. %, in another example from about 30 wt. % to about 55 wt. %, in another example from about 40 wt. % to about 30 wt. %, in another example from about 42 wt. % to about 48 wt. %, in another example from about 44 wt. % to about 46 wt. %, and in another example about 45 wt. %. the wt. % increase after the pH sensitive coating is added is from about 10 wt. % to about 50 wt. %, in another example from about 20 wt. % to about 45 wt. %, in another example from about 30 wt. % to about 40 wt. %, in another example from about 32 wt. % to about 38 wt. %, in another example from about 34 wt. % to about 36 wt. %, and in another example about 35 wt. %. In another example, the wt. % increase after the pH sensitive coating is added is from about 30 wt. % to about 50 wt. % and in another example from about 35 wt. % to about 45 wt. %.

In another example, the delayed release particles can optionally comprise from about a 5 wt. % to about a 55 wt. % pH sensitive coating, by weight of the particle, in another example from about a 10 wt. % to about a 45 wt. %, and in another example from about a 15 wt. % to about a 35 wt. %.

The pH sensitive coating can be an enteric coating. In one example, the pH sensitive coating can be degradable in the small intestine at a pH of at least 5.5 and in another example the pH coating can be degradable when the pH is at least 7.0. In any event, the pH sensitive coating can avoid degradation premature phenylephrine dissolution in the low pH in the stomach.

The pH sensitive coating can contain one or more polymers alone or in combination with water soluble or insoluble polymers. The pH sensitive coating can contain any chemically stable, biocompatible polymer. In one example, the pH sensitive coating has a molecular weight of from 100,000 g/mol to 600,000 g/mol, in another example 150,000 g/mol to 500,000 g/mol, in another example 200,000 g/mol to 400,000 g/mol, in another example 225,000 g/mol to 350,000 g/mol, and in another example 250,000 g/mol to 300,000 g/mol. The pH sensitive coating can be applied as a solution containing from about 10% to about 30% solids and in one example a solution containing about 20% solids.

Non-limiting examples of polymers can include cellulose esters and derivatives, acrylate copolymers, hypromellose acetate succinate, polyvinyl acetates and derivatives (commercially available as Kollicoat®, from BASF, Tarrytown, N.J.), shellac, and combinations thereof.

Non-limiting examples of cellulose esters and derivatives can include cellulose acetate phthalate, hydroxypropyl methylcellulose phthalate (HPMCP), hydroxypropyl methylcellulose acetate succinate, hydroxyethyl cellulose, cellulose acetate tetrahydrophthalate, cellulose acetate hexahydrophthalate, hydroxypropyl cellulose acetate succinate, and combinations thereof.

Non-limiting examples of acrylate copolymers can include methyl-methacrylate esters copolymerized with methacrylic acid, acrylic acid and esters copolymerized with methacrylic acid and esters, ammonio-containing acrylate copolymers, and combinations thereof.

In one example, the polymer can be an anionic copolymer based on methyl acrylate, methyl methacrylate, and methacrylic acid. In one example, the coating can contain Poly(methyl acrylate-co-methyl methacrylate-co-methacrylic acid) 7:3:1 polymer marketed under the tradename “Eudragit® FS30D”, available from Evonik Industries, Darmstadt, Germany. In another example, the coating can further comprise Poly(methacrylic acid-co-ethyl acrylate) 1:1 polymer, marketed under the tradename “Eudragit® L30D”, commercially available from Evonik, Darmstadt, Germany.

In one example, the pH sensitive coating can contain both Eudragit® FS30D and Eudragit® L30D. In one example, the pH sensitive coating can contain from 50% to 95% FS30D, by weight of the total Eudragit®, in another example 60% to 90%, and in another example 70% to 85%. In one example, the pH sensitive coating can contain 85% FS30D and 15% L30D by weight of the Eudragit®, in another example the pH sensitive coating can contain 90% FS30D and 10% L30D.

In one example, the pH sensitive coating can contain more than one polymer that can be mixed at any ratio to control where the phenylephrine is released.

In one example, the immediate release particles can have a polymer coating, which is not an enteric coating and can dissolve upon hitting the stomach.

In another example, the pH sensitive coating can contain a processing aid. Non-limiting examples of processing aids can include Plasacryl™ T20 (commercially available from Evonik), which can include a premix of polysorbate 80, triethyl citrate, and glycerol monostearate.

In another example, the pH sensitive coating can be colored. For instance, in one example the pH sensitive coating can contain a pigment and/or dye.

### Smoothness Test Method

The Smoothness Test Method can be used to determine the circularity of the particles. Circularity is determined by (4π×([Area])/([Perimeter]²) and ranges from 0 (infinitely elongated polygon) to 1 (perfect circle). Thus, a particle with a rough, coarse, or spiked appearance can have a larger perimeter value as compared to a smooth particle with the same area. Therefore, differences in surface topology can be calculated using the differences in the obtained circularity results.

Using a microscope (Nikon OPTIPHOT-2) and 40× magnification (4× magnifier and 10× eyepiece) and a digital camera (OptixCam Summit OCS-10.0) designed for microscopy, select the field of view that contains the particles to be analyzed. There should be spaces between the particles in the selected field of view.

The image is saved in an acceptable file format, such as JPEG, and opened using ImageJ 1.49v (Image Processing and Analysis in Java) computer software using the “File/Open” menu pointed to the stored file directory.

Next, adjust the settings on ImageJ. Open the threshold settings panel and select the following: method (Default), Color (B&W), and Color Space (HSB).

The next step is to tune the white background and black particles to make sure that the images to be studied are completely filled within the outline masks. This is done using the brightness sliders in the software program. Slide the brightness slider so snow appears in the background, as in FIG. 13A. Then, slide the brightness adjustments just until the background becomes white again, without any snow, as in FIG. 13B.

The image is ready for measurement processing. Using the “Set Measurements” menu, assign the measurements t be taken for the image. For this test, “Shape descriptors” must be checked for circularity and roundness measurements. Then, use the “Analyze Particles” command from the “Analyze” menu to select a size filter, to omit any small particles to not be included in measurement. This is done by selecting size (pixel̂2): 500-Infinity. In the “Analyze Particles” command, also select display results, clear results, summarize, exclude on edges, and include holes. Exclude on edges will not include any threshold particles on the edge of the image, only those within full view. Also select Show: “Overlay Outlines” to create new image with analyzed particles highlighted for easy reference. Now, select “OK” to analyze the particles. An image summary report and outline overlay of the original image will be displayed.

Repeat ten times with each population of particles, changing the field of view each time and calculate the mean circularity.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for forming a coated core comprising:
a. in a fluidized bed processor, discharging a spray comprising atomized air and a coating solution wherein the coating solution comprises an active;
b. wetting a core with the coating solution;
c. drying the wetted cores to form coated cores;
d. repeating steps a, b, and c until the % active on the coated core is from about 8% to about 30%, by weight of the coated core;
wherein the coated cores are substantially smooth as visually perceived under a microscope with a total magnification of 40×.

2. The process of claim 1 further comprising step (e) applying a pH sensitive coating and forming a delayed release particle.

3. The process of claim 2 wherein the pH sensitive coating is an enteric coating.

4. The process of claim 2 further comprising step (f) applying an anti-caking coating.

5. The process of claim 2 further comprising applying a separation coating after applying the active coating and before applying the pH sensitive coating.

6. The process of claim 1 wherein the % active on the coated core is from about 10% to about 25%, by weight of the coated core.

7. The process of claim 6 wherein the % active on the coated core is from about 13% to about 18%, by weight of the coated core.

8. The process of claim 1 wherein the fluidized bed comprises an air inlet wherein the air inlet has an inlet air dew point from about 7° C. to about 15° C.

9. The process of claim 1 wherein the active coating solution comprises from about 10% to about 40% active.

10. The process of claim 1 wherein the cores comprises a material selected from microcrystalline cellulose, sugars, starches, polymers, and combinations thereof.

11. The process of claim 1 wherein the cores have a diameter of about 500 μm to about 710 μm.

12. The process of claim 1 wherein the active comprises a freely soluble active.

13. The process of claim 1 wherein the active comprises phenylephrine hydrochloride.

14. A process for forming a coated core comprising:
a. in a fluidized bed processor, discharging a spray comprising atomized air and a coating solution wherein the coating solution comprises phenylephrine or a salt thereof;
b. wetting a core with the coating solution;
c. drying the wetted cores to form coated cores;
repeating steps a, b, and c until the core has a % weight increase from about 15% to about 25%; and
wherein the fluidized bed processor comprises an absolute humidity and wherein the absolute humidity is less than about 20 g of water vapor/kg of dry air.

15. The process of claim 14 wherein the coated cores comprise a circularity from about 0.7 to about 1 as determined by the Smoothness Test Method.

16. The process of claim 15 wherein the coated cores comprise a circularity from about 0.8 to about 1 as determined by the Smoothness Test Method.

17. The process of claim 14 wherein the fluidized bed processor comprises an absolute humidity and wherein the absolute humidity is less than about 18 g of water vapor/kg of dry air
